(19) United States
(12) Patent Application Publication
(10) Pub. No.: US 2015/0104527 A1
(43) Pub. Date: Apr. 16, 2015

(54) AQUEOUS IONIC SOLUTION CONTAINING SEAWATER AND AT LEAST ONE COMPOUND THAT IS ORIGINALLY IMMISCIBLE WITH SEA WATER

(71) Applicants: Francois Andro, Saint-Symphorien (FR); Anne Beaulieu, Dinard (FR)

(72) Inventors: Francois Andro, Saint-Symphorien (FR); Anne Beaulieu, Dinard (FR)

(73) Assignee: LABORATOIRE DE LA MER, Saint-malo (FR)

(21) Appl. No.: 14/361,218

(22) PCT Filed: Feb. 2, 2012
(86) PCT No.: PCT/EP2012/052886

§ 371 Date: Nov. 24, 2014

(30) Foreign Application Priority Data
Feb. 18, 2011 (FR) 1151357

(51) Int. Cl. A61K 47/24; A61K 36/534; A61K 36/61; A61K 33/20

(52) U.S. Cl. CPC A61K 47/24; A61K 33/20; A61K 36/534; A61K 36/61
USPC 424/663

(57) ABSTRACT

The invention relates to an aqueous ionic solution, particularly for nasal use, including: at least one seawater solution having an osmolality of between 290 mOsm/kg and 400 mOsm/kg in a weight proportion of between about 30 and 98 wt % relative to the total weight of the composition; at least one cationic phospholipid of natural origin in a weight proportion of between about 0.01 and 5 wt % relative to the total weight of the composition; and at least one compound, which is originally immiscible with said seawater solution, in a weight proportion of between 0.01 and 10 wt % relative to the total weight of the composition.

### 1. FIELD OF THE INVENTION

The field of the invention is that of solutions based on seawater, used in particular in the treatment of mucosae, in particular nasal, or more generally the ear, nose and throat sphere, or the skin.

### 2. PRIOR ART

Numerous solutions based on seawater are known in the prior art. “Solution based on seawater” means in the present description any solution containing more than 25% by weight seawater, preferably more than 75% by weight seawater, which has an osmolarity of between 250 mOsm/kg and 350 mOsm/kg, in particular between 290 mOsm/kg and 315 mOsm/kg. The osmolarity sought may be obtained either by depletion of seawater with regard to some ionic species, or by diluting seawater with pure water.

This definition does not include so-called physiological serum or saline solutions. These solutions are called thus since they are isotonic. However, they contain only Na and Cl ions by way of solely ionic species. Saline solutions or physiological serums are therefore very different from seawater, through their ionic composition and the quantity of ions contained.

The applicant company has already described the application of iso-osmotic ionic solutions based on seawater for preventing and limiting the release of chemical mediators responsible for triggering inflammatory phenomena in the bronchial and pulmonary mucosae, in particular in the patent EP 1 091 747, but also for cerumenolytic treatment, in the patent EP 1 091 746. The applicant has also described the use of seawater for treating nasal congestion by means of hypertonic solutions in the patent EP 2 068 896.

Moreover, the applicant company has developed aqueous ionic solutions of the type in question, enriched with potassium for use for treating and washing eyes as well as for use as a contact lens rinsing product. These solutions and the uses thereof are described in particular in the patents FR 2 843 029 and FR 2 803 205.

Seawater has the advantage of containing numerous oligoelements of natural origin, essential for maintaining cell balance. The solutions developed based on seawater, hereinafter referred to as “solutions based on seawater”, may be isotonic (with an osmolarity of between 250 mOsm/kg and 350 mOsm/kg approximately) or hypertonic (the osmolarity of which is above 350 mOsm/kg). Isotonic solutions are in particular used for their hydrating, reparatory and anti-inflammatory properties. Hypertonic solutions are in particular used for the decongestion of pro-inflammatory mucosae and fluidifying the secretions produced.

To reinforce or diversify the properties of a solution based on seawater, it may be advantageous to add to the solution compounds having other preventive or curative properties.

The patent FR 2 569 536 proposes, for preparing parenteral nutritive solutions, to bind seawater and vitamins with lecithin. This emulsifier is widely used in the food industry for emulsions binding water and lipids.

The patent application EP 1 074 245 proposes associating plant extracts with Dead Sea salts for dermatological applications.

### 3. DRAWBACKS OF THE PRIOR ART

Some compounds of natural or chemical origin that it would be advantageous to associate with solutions based on seawater, because of their beneficial properties with regard to the skin or mucosae, are however immiscible in such solutions.

This immiscibility may result in particular from the natural osmotic force of seawater and/or the lipophilic character of such compounds.

Thus many plant extracts, in particular essential oils, but also certain vitamins, are originally immiscible with seawater solutions. Some aqueous extracts of plants are also immiscible with such solutions based on seawater because of the natural osmotic force of seawater.

In practice, the association of a solution based on seawater and such compounds originally immiscible with such a solution leads to unstable compositions that are not solutions but at best emulsions that more or less quickly form precipitates, and/or unstable emulsions that dephase.

In addition, the compositions obtained by means of such associations may have pHs reaching non-physiological values, that is to say pHs either lower than 7 or higher than 8.4, which make them unusable or difficult to use for use in particular in the ear, nose and throat sphere.

### 4. OBJECTIVES OF THE INVENTION

An objective of the invention is in particular the association, in an aqueous ionic solution, of a solution based on seawater and at least one compound originally immiscible with such a solution based on seawater.

In particular, one objective of the invention is to propose such an aqueous ionic solution that is stable, that is to say that does not dephase and does not form precipitates.

Yet another objective of the invention is to propose such an ionic solution that has, in at least some embodiments, a pH of between 7 and 8.4.

Yet another objective of the invention is to disclose such an ionic solution which, in at least some embodiments, can be applied by atomisation or nebulisation.

Another objective of the invention is to provide such a composition which, in at least some embodiments, is clear.

Another objective of the invention is to use such a composition which, in at least some embodiments, can be used for treating, for preventive or curative purposes, the ear, nose and throat sphere, in particular the nasal mucosae, and/or for treating, for preventative or curative purposes, the dermatological zone, and/or for treating, for preventative or curative purposes, the gastrointestinal area, and/or for treating, for preventative or curative purposes, disorders related to nutrition.

Another objective of the invention is to propose, in at least one of its embodiments, an aqueous ionic solution for nasal use, comprising a solution based on seawater and at least one compound originally immiscible with such a solution based on seawater.

Yet another objective of the invention is to propose a method for preparing such a composition.

### 5. DISCLOSURE OF THE INVENTION

All or some of these objectives are achieved by means of, in accordance with the invention, an aqueous ionic solution, intended in particular for nasal use, comprising at least:
a solution based on seawater having an osmolarity lying, for an isotonic solution, between 250 mOsm/kg and 350 mOsm/kg, and for a hypertonic solution above 350 mOsm/kg and more particularly between 350 mOsm/kg and 1074 mOsm/kg, in a proportion by mass lying between approximately 25% and 98% with respect to the total mass of the composition,
at least one cationic phospholipid of natural origin in a proportion by mass lying between approximately 0.01% and 5% with respect to the total mass of said aqueous ionic solution,
at least one compound originally immiscible with said solution based on seawater in a proportion by mass lying between approximately 0.01% and 10% with respect to the total mass of said aqueous ionic solution.

According to the invention, the presence in an aqueous ionic solution of a cationic phospholipid of natural origin enables the originally immiscible compound to be solubilised in a solution based on seawater.

The inventors in fact discovered surprisingly that cationic phospholipids of natural origin had a solubilising capacity making it possible to associate a solution based on seawater and a compound originally immiscible with this solution based on seawater in order to form an aqueous ionic solution according to the invention. In addition, using a solubiliser of natural origin limits irritation and sensitisation of the nose and throat mucosae by chemical products. This point is particularly important when these mucosae are weakened by inflammation, infection, an allergic reaction or environmental pollution.

Such solutions according to the invention are stable. They may also be very clear. A stable solution means a solution the essential properties of which, in a given period of time, do not change or change at most in tolerable proportions. In addition it is understood that the solution must be stored under suitable prescribed conditions of temperature, humidity and exposure to light and that a suitable receptacle has been used.

The stability of the following properties is sought:
Chemical stability: active-principle content of between 95% and 105%
Physical stability: appearance, taste
Microbiological stability: contamination, proliferation
Therapeutic stability: therapeutic effect unchanged
Toxicological stability: no increase in toxicity (degradation of products) (Swiss Pharmacopeia, 9^{th} edition).

The limpidity of a solution may be measured by mirage: examined under appropriate conditions it must be free from particles visible to the naked eye (PE 2.9.20). Limpidity may also be measured by optical density using a spectrophotometer (PE 2.2.25 and PE 2.2.23).

The aqueous ionic solutions according to the invention also have the advantage of being able to be applied by atomisation or nebulisation.

An aqueous ionic solution according to the invention associating a solution based on seawater and at least one originally immiscible compound may reinforce the beneficial properties of the solution based on seawater.

Such properties may in particular be due to properties of hydration of the skin or mucosae, properties of repairing the skin or mucosae, anti-inflammatory properties for the skin and mucosae, properties of fluidising the nasal mucus and/or also properties for decongestion of the nasal mucosae.

An aqueous ionic solution according to the invention may make it possible to combine, or even potentialise, the properties of the solution based on seawater and those of the originally immiscible compound, in particular in the treatment of ailments in the ear, nose and throat sphere and the respiratory tract.

Another advantage of the composition according to the invention is to increase the duration of action of the solution based on seawater, by virtue of the presence of the originally immiscible compound.

Another advantage of the composition according to the invention may be to limit the use of medicinal specialities, such as vasoconstrictors or antiseptics.

Another advantage of the composition according to the invention is to improve observance by patients because of the agreeable sensation for the patient.

In particular, the aqueous ionic solution according to the invention may comprise:
said solution based on seawater having an osmolarity of between 290 mOsm/kg and 350 mOsm/kg, in particular between 290 mOsm/kg and 315 mOsm/kg in a proportion by mass of between approximately 90% and 98% with respect to the total mass of said aqueous ionic solution,
said at least one cationic phospholipid of natural origin, in a proportion by mass of between approximately 0.10% and 0.20% with respect to the total mass of said aqueous ionic solution,
said at least one compound originally immiscible with said solution based on seawater in a proportion by mass of between approximately 0.10% and 0.30% with respect to the total mass of said aqueous ionic solution.

The solution based on seawater may be like the ones described in the patent documents FR-A-2915389, EP-A-1091747, EP-A-1091746, FR-A-2843029, EP-A-2 068 896 and FR-A-2803205.

Said at least one compound originally immiscible with the solution based on seawater may be of natural origin or chemical origin. Said at least one compound originally immiscible with said solution based on seawater may be chosen from the group consisting of: aqueous or oily extracts of plants, solutions comprising at least one lipophilic vitamin and mixtures thereof.

Said compound originally immiscible in the solution based on seawater may be chosen according to the intended use of the composition, for example according to the desired therapeutic or preventive effect. It may chosen for example from plant extracts, in particular plant essential oils such as essential oil of ravintsara, essential oil of mint, essential oil of sage, essential oil of lemon, essential oil of thyme, essential oil of rosemary, essential oil of pine, essential oil of gaultheria, essential oil of myrtle, essential oil of myrrh, etc. In particular, said compound originally immiscible with said solution based on seawater may comprise an oily extract of mint, eucalyptus and/or niaouli.

Said compound originally immiscible in the solution based on seawater may also be chosen from vitamins, in particular A, D, E and K, or fatty acids such as sweet almond oil, olive oil or apricot kernel oil. Moisturisers such as beeswax and glycerine can also be cited by way of example as a compound originally immiscible in the solution based on seawater.

The cationic phospholipid of natural origin preferentially contains myristamidopropyl propylene glycol dimonium chloride phosphate, which may advantageously be extracted from the coconut. The cationic phospholipid of natural origin may also comprise phosphatidylcholine and/or cationic phospholipids issuing from milk.

The aqueous ionic solution according to the invention may also comprise a natural preservative in a proportion by mass lying between approximately 0.01% and 10% with respect to the total mass of said aqueous ionic solution, said natural preservative being able to withstand sterilisation in a wet environment at 120° C. for approximately 20 minutes. Said natural preservative may in particular be in a proportion by mass lying between approximately 0.10% and 0.80% with respect to the total mass of said aqueous ionic solution. Said natural preservative may comprise for example a homopolymer of L-lysine.

The aqueous ionic solution according to the invention preferably does not comprise any chemical preservative, the repeated or prolonged use of which may be harmful to the physiology, in particular the nasal mucus membrane. This is because the chemical preservative most used currently is benzalkonium chloride. However, it has been described in the literature that this preservative of chemical origin causes deleterious effects on the nasal mucus membrane. More precisely, benzylkonium chloride is considered to be toxic for human neutrophiles, even at low concentration (Boston et al, 2003, Arch. Otolaryungolo Head Neck Surgery, vol. 129, 660-664). This effect of benzylkonium chloride on the nasal mucus membrane has been observed in patients even for short use (Graf P et al, 1999, Arch Otolaryngolo Head Neck Surgery, vol. 125, 1128-1132). The composition according to the invention therefore solves this problem by incorporating a natural preservative.

The aqueous ionic solution may also comprise a refreshing compound, in a proportion by mass lying between approximately 1% and 10%, in particular between approximately 1% and 4%, with respect to the total mass of said aqueous ionic solution. According to one embodiment, said refreshing compound may comprise sorbitol, xylitol, menthol, etc.

The aqueous ionic solution according to the invention may for example be used for manufacturing lotions, wipes, sprays or the like.

Another subject matter of the invention, in combination with the above, is the use of the aqueous ionic solution as defined above for treating the nasal mucosae.

Another subject matter of the invention, in combination with the above, is the use of an aqueous ionic solution as defined above for manufacturing a medication or a product for pharmaceutical use intended to prevent or treat ailments of the ear, nose and throat sphere. The common cold, acute rhinosinusitis, acute sinusitis, chronic rhinosinusitis, allergic rhinitis, otitis, rhinopharyngitis, pharyngitis, laryngitis, tracheitis and bronchitis can be cited by way of example as ailments of the ear, nose and throat sphere.

Another subject matter of the invention, in combination with the above, is the use of the aqueous ionic solution as defined above for treating dermatological ailments, ailments of the gastrointestinal sphere and/or ailments relating to nutrition. Another subject matter of the invention, in combination with the above, is the method for preparing the aqueous ionic solution as defined above.

The method for preparing the aqueous ionic solution according to the invention may be as follows:

A solution based on seawater having an osmolarity of between 250 mOsm/kg and 350 mOsm/kg, in particular between 290 mOsm/kg and 315 mOsm/kg approximately, the osmolarity being able to be chosen according to the required use of the aqueous ionic solution, is prepared as described below.

Each of the raw materials consisting of the solubiliser or solubilisers, the compound or compounds originally immiscible in seawater and the refreshing compound or compounds, where applicable, are homogenised.

These raw materials are weighed.

A first solubiliser is put in a vessel and then, incorporated under stirring, a second solubiliser where applicable solubilising the compound or compounds originally immiscible, such as essential oils, optionally the natural preservative and optionally the refreshing compound or aroma. The mixing is carried out for approximately 15 minutes.

This mixture of raw materials is assembled with the solution based on seawater and mixing is carried out for approximately 30 minutes in order to form the aqueous ionic solution according to the invention.

Advantageously, the first solubiliser is myristamidopropyl propyleneglycol dimonium chloride phosphate and the second solubiliser, sorbitol. The use of sorbitol as the second solubiliser avoids having to add excessively heavy concentrations of myristamidopropyl propyleneglycol, which could prove irritating for fragile mucosae.

### 6. EXAMPLES

### 6.1. Hypertonic Solution Based on Seawater with Essential Oils of Mint and Eucalyptus

An aqueous ionic solution having the following composition is produced (proportions by mass with respect to the total mass of the aqueous ionic solution):
Aqueous hypertonic solution based on seawater, with a salt content of 22 g/l, electrodialysed: 97.54%
Epsilon-polylysine (polypeptide the lysines of which are bonded together by epsilon bonds): 0.2%
Sorbitol: 0.16%
Myristamidopropyl propyleneglycol dimonium chloride phosphate: 2%
Mint-eucalyptus aroma LN 05614/1 (42.2% vol): 0.1%
   In this example, the aroma contains by volume:
ethyl alcohol at 96%,
monopropyleneglycol,
essential oil of eucalyptus: 0.042%, and
crystallised menthol extract of Mentha arvensis: 0.0112%

To produce this aqueous ionic solution, the following procedure is used.

A solution based on seawater with a salt content of 22 g/l is prepared by electrodialysis, having an osmolarity of between 310 mOsm/kg and 325 mOsm/kg for the isotonic solution, and beyond 350 mOsm/kg for a hypertonic solution. The method for producing this solution based on seawater, known from the prior art, is stated below.

Each of the raw materials consisting in this example of epsilon polylysine, myristamidopropyl propyleneglycol dimonium chloride phosphate, sorbitol and mint-eucalyptus aroma, was homogenised.

Each of these raw materials was weighed, and the myristamidopropyl propyleneglycol dimonium chloride phosphate was placed in a vessel and then, under stirring, the epsilon-polylysine, the sorbitol and the mint-eucalyptus aroma.

After the whole was mixed for approximately 15 minutes, it was added to the solution based on seawater in order to form the required aqueous ionic solution after mixing for approximately 30 minutes. The solution is perfectly clear.

### 6.2. Hypertonic Solution Based on Seawater with Essential Oils and Mint, Eucalyptus and Niaouli

An aqueous ionic solution having the following composition is prepared (proportions by mass with respect to the total mass of the aqueous ionic solution):
Aqueous hypertonic solution based on seawater, with a salt content of 22 g/l, electrodialysed: 90%
Epsilon-polylysine (polypeptide the lysines of which are bonded together by epsilon bonds): 8%
Sorbitol: 1.55%
Myristamidopropyl propyleneglycol dimonium chloride phosphate: 0.1%
Mint-eucalyptus aroma LN 05614/1 (42.2% vol): 0.2%
Essential oil of niaouli (Melaleuca quinquenervia): 0.1%
Ethyl alcohol 96% PE (Ethanol 96 superfine): 0.05%.
   In this example, the aroma contains by volume
ethyl alcohol at 96%,
monopropyleneglycol,
essential oil of eucalyptus: 0.042%, and
crystallised menthol extract of Mentha arvensis: 0.0112%

This solution was prepared according to the same operating method described in the previous example. The solution is perfectly clear.

### 6.3. Hypertonic Solution Based on Seawater with Essential Oils of Thyme and Lemon

An aqueous ionic solution having the following composition is prepared (proportions by mass with respect to the total mass of the aqueous ionic solution):
Hypertonic aqueous solution based on seawater, with a salt content of 22 g/l, electrodialysed: qsp 100%
Sorbitol (Neosorb 70/70B): 2%
Myristamidopropyl propyleneglycoldimonium chloride phosphate: 0.3%
Essential oil Thgymus zygis L: 0.1%
Essential oil of lemon: 0.054%.

This solution was prepared according to the same operating method described in the previous example. The solution is perfectly clear.

### 7. The Solution Based on Seawater

As described in the patent application FR-A-2915389, the solution based on seawater used in the above formulations, which is an iso-osmotic ionic solution based on seawater, may have:
a pH of between 7.6 and 8.4,
a dry matter content of 1% to 2% by weight,
an osmolarity of between 250 and 400 mOsm/kg, in particular between 250 mOsm/kg and 350 mOsm/kg approximately,
500 to 2600 mg/l of sodium (Na),
40 to 6500 mg/l of potassium (K),
5800 to 7000 mg/l of chloride (Cl),
20 to 400 mg/l of calcium (Ca),
50 to 1500 mg/l of magnesium (Mg).

The solution based on seawater may be sterile or be sterilised.

The solution based on seawater may comprise other elements, such as iron (Fe), zinc (Zn), copper (Cu), manganese (Mn) or selenium (Se).

The solution based on seawater is prepared by electrodialysis of seawater.

More particularly, successively:
there is taken off, as raw material, seawater, with a salt content greater than or equal to 32 g/l, preferably at a depth of 5 to 10 metres in an area with strong current movements,
this water is analysed and clarified,
the settled water has salt removed by electrodialysis until an osmolarity of between 290 mOsm/kg and 350 mOsm/kg is obtained, in particular between 290 mOsm/kg and 315 mOsm/kg for an isotonic solution beyond 350 mOsm/kg and more particularly between 350 mOsm/kg and 1074 mOsm/kg for a hypertonic solution,
the ionic concentrations are adjusted by selective electrodialysis,
the product is filtered and stored, optionally under sterile conditions.

Throughout the present description, the expression “comprising a” must be understood as being synonymous with the expression “comprising at least one”, unless the contrary is specified.

Throughout the present description, the ranges of values must be understood as including the bounds, unless the contrary is specified.

### 8. Counter-Examples

Tests carried out under the same conditions but using other types of solubiliser instead of a cationic phospholipid of natural origin according to the invention led to unstable compositions not forming solutions or having drawbacks making them unusable in the context of the envisage applications. Limpidity was measured by optical density using a spectrophotometer (PE 2.2.25 and PE 2.2.23).

For example, the solubiliser DISPER®, which is an emulsifying complex for essential oils, flocculates in contact with seawater. The surfactant SoluBol® tends to precipitate and consequently no longer fulfils its role. MontanOV® gives a cloudy solution while the use of glycerine as a solubiliser gives an opalescent solution. However, it is necessary to obtain a perfectly clear solution for the pleasure of use. On the other hand, the solutions prepared in accordance with the examples indicated in the present application are perfectly clear.

1. Aqueous ionic solution, in particular intended for nasal use, comprising at least:
a solution based on seawater having an osmolarity lying, for an isotonic solution, between 250 mOsm/kg and 350 mOsm/kg or, for a hypertonic solution, an osmolarity above 350 mOsm/kg, in a proportion by mass lying between approximately 25% and 98% with respect to the total mass of said aqueous ionic solution,
at least one cationic phospholipid of natural origin in a proportion by mass lying between approximately 0.01% and 5% with respect to the total mass of said aqueous ionic solution,
at least one compound originally immiscible with said solution based on seawater in a proportion by mass lying between approximately 0.01% and 10% with respect to the total mass of said aqueous ionic solution.

2. Aqueous ionic solution according to claim 1, characterised in that it comprises at least:
said solution based on seawater having an osmolarity of between 290 mOsm/kg and 350 mOsm/kg for an isotonic solution or an osmolarity greater than 350 mOsm/kg for a hypertonic solution, in a proportion by mass between approximately 90% and 98% with respect to the total mass of said aqueous ionic solution,
said at least one cationic phospholipid of natural origin, in a proportion by mass of between approximately 0.10% and 0.20% with respect to the total mass of said aqueous ionic solution,
said at least one compound originally immiscible with said solution based on seawater in a proportion by mass of between approximately 0.10% and 0.30% with respect to the total mass of said aqueous ionic solution.

3. Aqueous ionic solution according to one of claims 1 and 2, characterised in that said at least one compound originally immiscible with said solution based on seawater is chosen from the group consisting of: aqueous or oily extracts of plants, the solutions comprising at least one lipophilic vitamin and mixtures thereof.

4. Aqueous ionic solution according to one of claims 1 to 3, characterised in that it comprises a natural preservative in a proportion by mass lying between approximately 0.01% and 10% with respect to the total mass of the composition, said natural preservative being able to withstand sterilisation in a wet environment at 120° C. for approximately 20 minutes.

5. Aqueous ionic solution according to claim 4, characterised in that said natural preservative is in a proportion by mass of between approximately 0.10% and 0.80% with respect to the total mass of the composition.

6. Aqueous ionic solution according to one of claims 4 and 5, characterised in that said natural preservative comprises a homopolymer of L-lysine.

7. Aqueous ionic solution according to any one of claims 1 to 6, characterised in that it comprises a refreshing compound, in a proportion by mass of between approximately 1% and 10%, in particular between approximately 1% and 4%, with respect to the total mass of the composition.

8. Aqueous ionic solution according to claim 7, characterised in that said refreshing compound comprises sorbitol.

9. Aqueous ionic composition according to any one of claims 1 to 8, characterised in that said compound originally immiscible with said solution based on seawater comprises an oily extract of mint and eucalyptus.

10. Use of the aqueous ionic solution according to any one of claims 1 to 9 for manufacturing a medication or a product for pharmaceutical use intended to prevent or treat ailments in the ear, nose and throat sphere.
